(19) United States
(12) Patent Application Publication
(10) Pub. No.: US 2008/0280926 A1
(43) Pub. Date: Nov. 13, 2008

(54) Phosphodiesterase Inhibitors

(76) Inventors: Venkata P. Palle, Haryana (IN); Sarala Balachandran, Delhi (IN); Mohammad Salman, Princeton, NJ (US); Gagan Kukreja, Delhi (IN); Lalit Kumar Baregama, Rajasthan (IN); Abhijit Ray, Delhi (IN); Sunanda Ghosh Dastidar, Delhi (IN)

Correspondence Address: RANBAXY INC., 600 COLLEGE ROAD EAST, SUITE 2100, PRINCETON, NJ, 08540, US

(21) Appl. No.: 10/596,528

(22) PCT Filed: Dec. 16, 2004
(86) PCT No.: PCT/IB04/04173

§ 371 Date: May 1, 2008

Related U.S. Application Data
(60) Provisional application No. 60/529,824, filed on Dec. 16, 2003.

(30) Foreign Application Priority Data
Dec. 16, 2003 (US) 60/529824

(51) Int. Cl. C07D 473/34; A61K 31/52; A61P 37/00; A61P 29/00; A61P 31/12; A61P 11/00; A61P 19/02; A61P 1/00

(52) U.S. Cl. 514/263.2; 544/277

(57) ABSTRACT

The present invention relates to purine derivatives, which can be used as selective phosphodiesterase (PDE) type IV inhibitors. Compounds disclosed herein can be useful in the treatment of asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans. Also provided are processes for the preparation of disclosed compounds, pharmaceutical composition containing the disclosed compounds and their use as selective phosphodiesterase (PDE) type IV inhibitors.

### FIELD OF THE INVENTION

The present invention relates to purine derivatives, which can be used as selective phosphodiesterase (PDE) type IV inhibitors. Compounds disclosed herein can be useful in the treatment of asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans. Processes for the preparation of disclosed compounds, pharmaceutical composition containing the disclosed compounds and their use as selective phosphodiesterase (PDE) type IV inhibitors are provided.

### BACKGROUND OF THE INVENTION

It is known that cyclic adenosine-3′,5′-monophosphate (cAMP) exhibits an important role of acting as an intracellular secondary messenger (E. W. Sutherland, and T. W. Roll, Pharmacol. Rev., (1960) 12, 265). Intracellular hydrolysis of cAMP to adenosine 5′-monophosphate (AMP) causes a number of inflammatory diseases or conditions, for example, psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis. The most important role in the control of cAMP (as well as of cGMP) levels is played by cyclic nucleotide phosphodiesterase (PDE), which represents a biochemically and functionally, highly variable superfamily of the enzyme; eleven distinct families with more than 15 gene products are currently recognized. Although PDE 1, PDE 2, PDE 3, PDE 4, and PDE 7 all use cAMP as a substrate, only the PDE 4 and PDE VII types are highly selective for hydrolysis of cAMP. Inhibitors of PDE, particularly the PDE 4 inhibitors, such as rolipram or Ro-1724, are therefore known as cAMP-enhancers. Immune cells contain type IV and type III PDE, the PDE type IV being prevalent in human mononuclear cells. Thus, the inhibition of phosphodiesterase type IV has been a target for modulation and, accordingly, therapeutic invention in a range of disease processes.

The initial observation that xanthine derivatives, such as theophylline or caffeine, inhibit the hydrolysis of cAMP led to the discovery of the required hydrolytic activity in the cyclic nucleotide phosphodiesterase (PDE) enzymes. More recently, distinct classes of PDE have been recognized (J. A. Bervo and D. H. Reifsnyder, TIPS (1990) 11, 150) and their selective inhibition has led to improved drug therapy (C. D. Nicholus, R. A. Challiss and M. Shahid, TIPS (1991) 12, 19). Thus, it was recognized that inhibition of PDE IV could lead to inhibition of inflammatory mediator release (M. W. Verghese et. al, J. Mol. Cell. Cardiol., (1989) 12 (Suppl.II), S 61) and airway smooth muscle relaxation (T. J. Trophy in Directions for new Anti-Asthma Drugs, eds S. R. O'Donnell and (G. A. Perssan, (1988) 37, Birkheuserverlag).

WO 03/002566 discloses purine derivatives as A2B adenosine receptor antagonists. WO 01/44260 discloses particular purines and uses of these compounds for the treatment of bone related disorders and cancer. WO 01/49688 discloses purine derivatives, process for their preparation and use thereof. WO 01/02400 and EP 1,221,444 disclose fused imidazole compounds and treatments of diabetes mellitus. WO 99/11643 discloses heterocyclyl-substituted ring-fused pyridines and pyrimidine as corticotropin releasing hormone (CRH) antagonists, said to be useful for treating CNS and stress-related disorders. WO 03/11864 discloses the preparation of triazolylimidazopyrimidines and triazolylimidazopyridines as antagonists of adenosine A2 receptor for treatment of Parkinson's disease. WO 96/06845 discloses the preparation of substituted 9-alkyladenines as adenosine A1 receptor inhibitors. WO 01/00587 discloses the preparation of azolylbenzamides and analogues for treating osteoporosis.

European Patent No. 544445 discloses the preparation of furyl-substituted purines, oxazolopyrimidines and pteridines as adenosine antagonists. Japanese Patent No. 2002155082 discloses the process for preparing adenine derivatives. U.S. Pat. No. 6,028,076 discloses purine derivatives, which are useful for the treatment of cancer or viral diseases. U.S. Pat. No. 5,723,468 discloses the preparation of imidazopyridines and analogs as muscarinic agonists. U.S. Pat. No. 6,130,333 discloses the preparation of benzodioxolylbenzimidazoles and related compounds as phosphodiesterase inhibitors. U.S. Pat. Nos. 6,228,859 and 6,413,975 disclose purine derivatives described as having phosphodiesterase IV inhibitory activity. Biochem. and Biophys. Res. Comm., 288, 427-434 (2001) discloses 9-benzyladenine derivatives with selective phosphodiesterase-4 inhibiting properties.

However, there remains a need for novel purine derivatives useful as selective phosphodiesterase type IV inhibitors.

### SUMMARY OF THE INVENTION

Generally provided herein are purine derivatives, which inhibit the PDE-IV enzyme and thus can be used for the treatment of asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases. Processes for the synthesis of these compounds are provided herein. Pharmaceutically acceptable salts, pharmaceutically acceptable solvates, enantiomers, diastereomers or N-oxides of these compounds having the same type of activity are also provided.

Also provided are pharmaceutical compositions containing the compounds disclosed herein, which can also contain pharmaceutically acceptable carriers or diluents. Such pharmaceutical compositions can be used for the treatment of asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases.

Provided herein are compounds having the structure of Formula I,

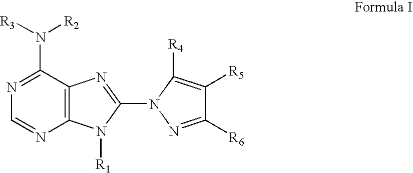

their pharmaceutically acceptable salts, pharmaceutically acceptable solvates, enantiomers, diastereomers or N-oxides wherein
R₁ can be hydrogen, alkyl, cycloalkyl, aryl, alkaryl, heteroaryl, heteroaryl alkyl, or heterocyclyl alkyl;
R₂ and R₃ independently are hydrogen, alkyl, alkenyl, alkynyl, acyl, alkaryl, heteroaryl alkyl, or heterocyclyl alkyl;
R₂ and R₃ together join to form three to eight membered cyclic rings, which can be optionally benzofused containing 0-3 heteroatom(s) selected from O, S or N, wherein the ring can be optionally substituted with one or more substituents selected from alkyl, alkenyl, alkynyl, cycloalkyl, carboxy, alkoxy, aryloxy, halogen, aryl, amino, substituted amino, alkaryl, heteroaryl, heterocyclyl, heteroarylalkyl or heterocyclyl alkyl; and
R₄, R₅ and R₆ are independently selected from hydrogen alkyl, aryl, heteroaryl, heterocyclyl, alkenyl, alkynyl, halogen, nitro, cyano, hydroxy, alkoxy, thioalkoxy, amino, or substituted amino;
   with the provisos that when R₂ is hydrogen, R₃ cannot be hydrogen, alkaryl or heteroaryl alkyl; when R₂ is alkyl, R₃ cannot be alkaryl or heteroaryl alkyl; when R₂ is alkaryl, R₃ cannot be hydrogen or alkyl; when R₂ is heteroaryl alkyl, R₃ cannot be alkyl; when R₁ is alkyl, R₂ and R₃ cannot be hydrogen and alkyl, respectively; and when R₁ is hydrogen; R₂ and R₃ cannot be hydrogen and alkyl, respectively.

In one aspect, R₁ can be aralkyl, for example, benzyl, 2-chlorobenzyl, 2-fluorobenzyl or 2-methoxybenzyl. In another aspect, R₂ can be hydrogen, acyl or aralkyl, for example, acetyl, benzoyl or 2-chlorobenzyl. In yet another aspect, R₃ can be alkyl, acyl or aralkyl, for example, methyl, ethyl, COCH₃, COC(CH₃)₃, COC₆H₅, CONH(4-chlorophenyl), CONHCH₂CH═CH₂ or 2-chlorobenzyl. In other aspects, R₄, R₅ and R₆ are hydrogen.

Also provided herein are compounds selected from:
N-(9-Benzyl-8-pyrazol-1-yl-9H-purin-6-yl)-2,2-dimethylpropionamide,
N-Acetyl-N-(9-benzyl-8-pyrazol-1-yl-9H-purin-6-yl)acetamide,
N-benzoyl-N-(9-benzyl-8-pyrazol-1-yl-9H-purin-6-yl)benzamide,
Bis-(2-chlorobenzyl)-[9-(2-chlorobenzyl)-8-pyrazole-1-yl-9H-purin-6-yl]-amine,
(9-Benzyl-8-pyrazol-1-yl-9H-purin-6-yl)methylamine,
1-(9-Benzyl-8-pyrazol-1-yl-9H-purin-6-yl)-3-(4-chlorophenyl)urea,
1-Allyl-3-(9-benzyl-8-pyrazol-1-yl-9H-purin-6-yl)-urea,
[9-(2-Methoxybenzyl)-8-pyrazol-1-yl-9H-purin-6-yl]-methylamine,
[9-(2-Fluorobenzyl)-8-pyrazol-1-yl-9H-purin-6-yl]-methylamine,
(9-Benzyl-8-pyrazol-1-yl-9H-purin-6-yl)ethylamine or
   their pharmaceutically acceptable salts, pharmaceutically acceptable solvates, enantiomers, diastereomers or N-oxides.

Also provided herein are pharmaceutical compositions comprising a therapeutically effective amount of at least one compound disclosed herein together with at least one pharmaceutically acceptable carrier, excipient or diluent.

Also provided are methods for treating, preventing, inhibiting or suppressing an inflammatory condition or disease in a patient, comprising administering to the said patient a therapeutically effective amount of at least one compound or pharmaceutical composition disclosed herein.

Further provided are methods for the treatment, prevention, inhibition or suppression of AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis or other inflammatory diseases in a patient comprising administering to said patient a therapeutically effective amount of at least one compound or pharmaceutical composition disclosed herein.

Also provided herein are methods for the preparation of compounds of Formula VII,

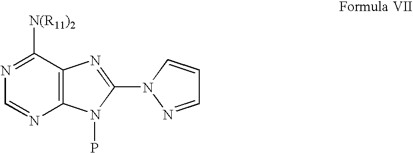

their pharmaceutically acceptable salts, pharmaceutically acceptable solvates, enantiomers, diastereomers or N-oxides, which method comprises the steps of:
a) N-protecting a compound of Formula II

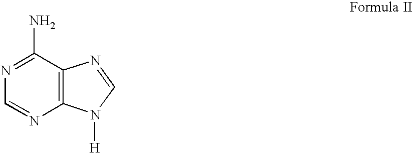

with a compound of Formula P-L to form a compound of Formula III,

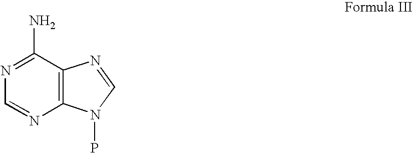

b) halogenating a compound of Formula III to form a compound of Formula IV,

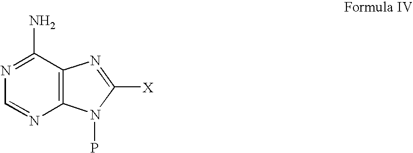

c) reacting a compound of Formula IV with pyrazole to form a compound of Formula VI,

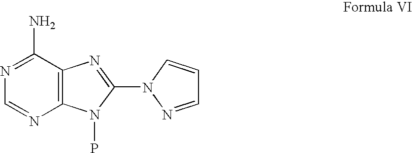

and
d) reacting a compound of Formula VI with a compound of Formula R₁₁-L to form a compound of Formula VII,
wherein P can be a protecting group; L can be a leaving atom or group; X can be a halogen; and R₁₁ can be R₃ (wherein R₃ can be hydrogen, alkyl, alkenyl, alkynyl, acyl, alkaryl, heteroaryl alkyl, or heterocyclyl alkyl).

Further provided herein are methods for the preparation of compounds of Formula XI,

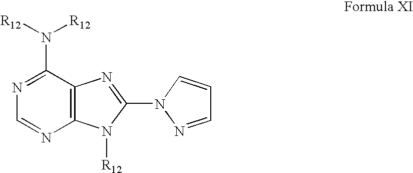

their pharmaceutically acceptable salts, pharmaceutically acceptable solvates, enantiomers, diastereomers or N-oxides, which method comprises the steps of:
a) deprotecting a compound of Formula VI

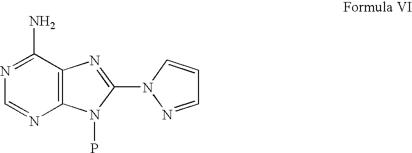

to form a compound of Formula VIII,

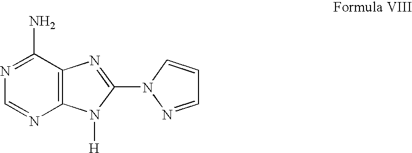

and
b) reacting a compound of Formula VIII with a compound of Formula R₁₂-L to form a compound of Formula XI
wherein P can be a protecting group, L can be a leaving atom or group and R₁₂ can be aralkyl.

Also provided are methods for the preparation of compounds of Formula XII,

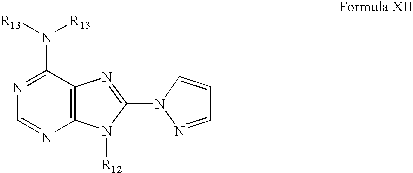

their pharmaceutically acceptable salts, pharmaceutically acceptable solvates, enantiomers, diastereomers or N-oxides, which method comprises the steps of:
a) reacting a compound of Formula VIII,

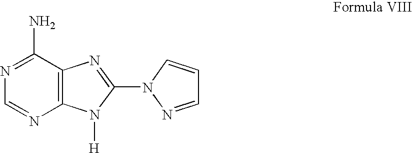

with a compound of Formula R₁₂-L to give a compound of Formula IX,

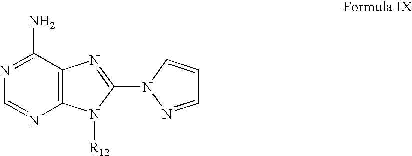

and
b) reacting a compound of Formula IX with a compound of Formula R₁₃-L to form a compound of Formula XII,
wherein L can be a leaving atom or group, R₁₂ can be aralkyl and R₁₃ can be R₂ or R₃ (wherein R₂ or R₃ independently can be hydrogen, alkyl, alkenyl, alkynyl, acyl, alkaryl, heteroaryl alkyl, or heterocyclyl alkyl).

Also provided herein are methods for the preparation of compounds of Formula XIII,

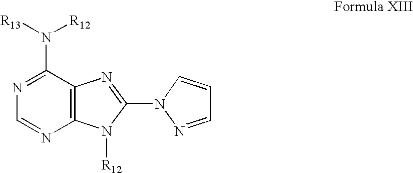

their pharmaceutically acceptable salts, pharmaceutically acceptable solvates, enantiomers, diastereomers or N-oxides, which method comprises the steps of:
a) reacting a compound of Formula VIII,

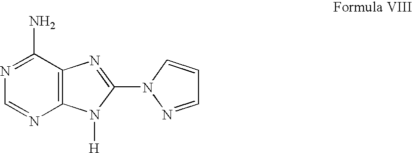

with a compound of Formula R₁₂-L to form a compound of Formula X,

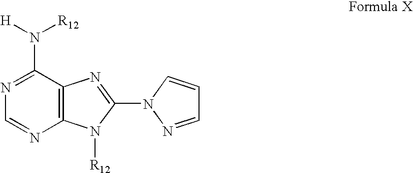

b) reacting a compound of Formula X with a compound of Formula R₁₃-L to form a compound of Formula XII,
wherein L can be a leaving atom or group, R₁₂ can be aralkyl, and R₁₃ can be R₂ or R₃ (wherein R₂ or R₃ independently can be hydrogen, alkyl, alkenyl, alkynyl, acyl, alkaryl, heteroaryl alkyl, or heterocyclyl alkyl).

Further provided herein are methods for the preparation of compounds of Formula

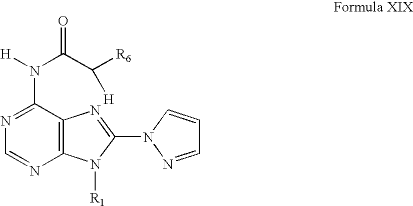

their pharmaceutically acceptable salts, pharmaceutically acceptable solvates, enantiomers, diastereomers or N-oxides, which method comprises the steps of:
a) reacting a compound of Formula III

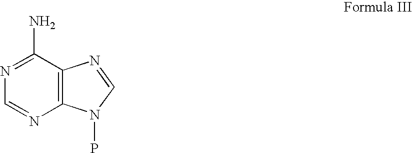

with a compound of Formula XIV,

R₆—NCO Formula XIV

to form a compound of Formula XV,

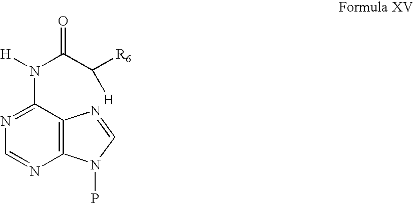

b) halogenating a compound of Formula XV to form a compound of Formula XVI,

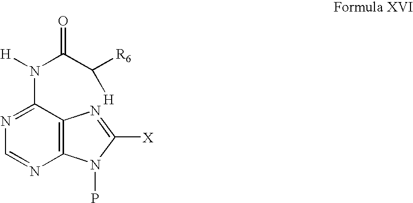

c) reacting a compound of Formula XVI with pyrazole gives a compound of Formula XVI,

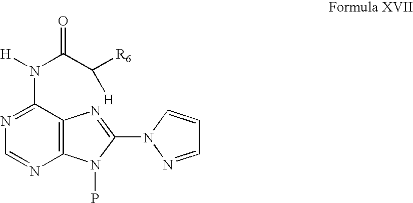

d) deprotecting a compound of Formula XVII to form a compound of Formula XVIII,

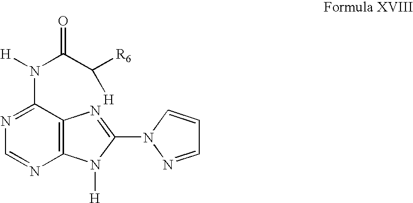

and
e) reacting a compound of Formula XVIII with a compound of Formula R₁-L to form a compound of Formula XIX,
wherein P can be a protecting group; R₆ can be hydrogen alkyl, aryl, heteroaryl, heterocyclyl, alkenyl, alkynyl, halogen, nitro, cyano, hydroxy, alkoxy, thioalkoxy, amino, or substituted amino; X can be a halogen; L can be leaving atom or group; and R₁ can be hydrogen, alkyl, cycloalkyl, aryl, alkaryl, heteroaryl, heteroaryl alkyl, or heterocyclyl alkyl.

Also provided herein are methods for the preparation of compounds of Formula XXIII,

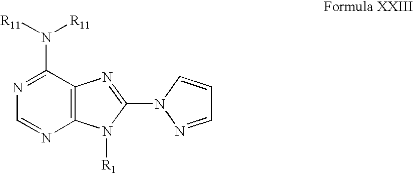

their pharmaceutically acceptable salts, pharmaceutically acceptable solvates, enantiomers, diastereomers or N-oxides, which method comprises the steps of:
a) reacting a compound of Formula III with a compound of Formula R₁₁-L

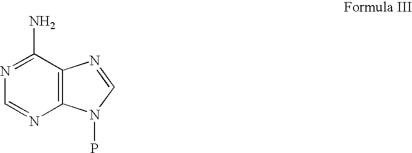

to form a compound of Formula VIIa,

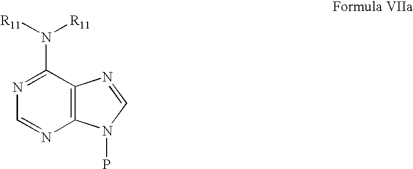

b) halogenating a compound of Formula VIIa to form a compound of Formula XX,

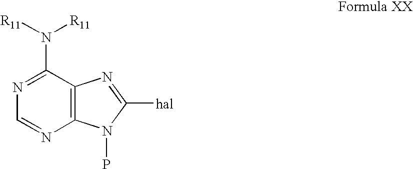

c) reacting a compound of Formula XX with pyrazole to form a compound of Formula XXI,

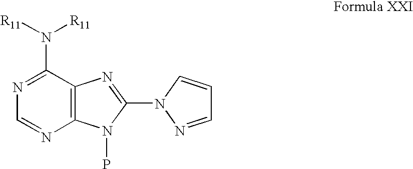

d) deprotecting a compound of Formula XXI to form a compound of Formula XXI,

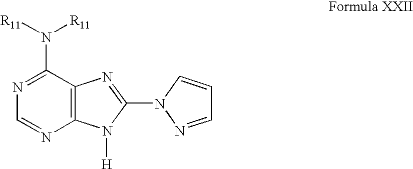

and
e) reacting a compound of Formula XXII with a compound of Formula R₁-L to form a compound of Formula XXIII,
wherein P can be a protecting group; L can be leaving atom or group; R₁ can be R₃ (wherein R₃ can be hydrogen, alkyl, alkenyl, alkynyl, acyl, alkaryl, heteroaryl alkyl, or heterocyclyl alkyl); hal can be halogen; and R₁ can be hydrogen, alkyl, cycloalkyl, aryl, alkaryl, heteroaryl, heteroaryl alkyl, or heterocyclyl alkyl.

Also provided herein are methods for the preparation of compounds of Formula XXIX,

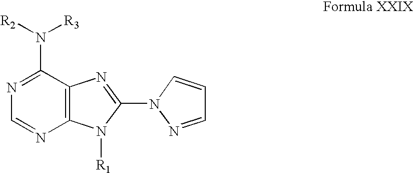

their pharmaceutically acceptable salts, pharmaceutically acceptable solvates, enantiomers, diastereomers or N-oxides, which method comprises the steps of:
a) reacting a compound of Formula XXIV

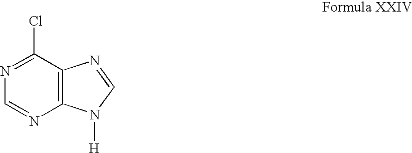

with a compound of Formula R₂R₃NH to form a compound of Formula XXVI,

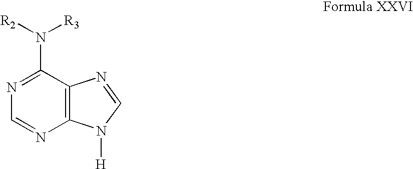

b) reacting a compound of Formula XXVI with a compound of Formula R₁-L to form a compound of Formula XXVII,

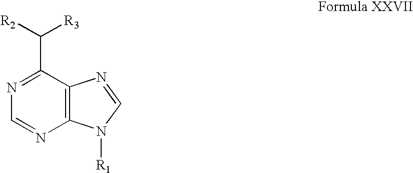

c) halogenating a compound of Formula XXVII to form a compound of Formula XXVIII,

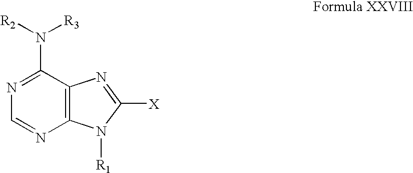

and
d) reacting a compound of Formula XXVIII with pyrazole to form a compound of Formula XXIX
wherein R₁ can be hydrogen, alkyl, cycloalkyl, aryl, alkaryl, heteroaryl, heteroaryl alkyl, or heterocyclyl alkyl; and R₂ and R₃ independently can be hydrogen, alkyl, alkenyl, alkynyl, acyl, alkaryl, heteroaryl alkyl, or heterocyclyl alkyl; L can be a leaving atom or group; and X can be a halogen.

While the present invention has been described in terms of its specific embodiments, certain modifications and equivalents will be apparent to those skilled in the art and are included within the scope of the present invention. Other aspects will be set forth in accompanying description which follows and in part will be apparent from the description or can be learnt by the practice of the invention.

### DETAILED DESCRIPTION OF THE INVENTION

In accordance with one aspect, there are provided compounds having the structure of Formula I,

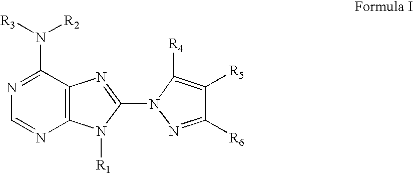

their pharmaceutically acceptable salts, pharmaceutically acceptable solvates, enantiomers, diastereomers or N-oxides wherein
R₁ can be hydrogen, alkyl, cycloalkyl, aryl, alkaryl, heteroaryl, heteroaryl alkyl, or heterocyclyl alkyl;
R₂ and R₃ independently can be hydrogen, alkyl, alkenyl, alkynyl, acyl, alkaryl, heteroaryl alkyl or heterocyclyl alkyl;
R₂ and R₃ can together join to form three to eight membered cyclic rings, which can be optionally benzofused containing 0-3 heteroatom(s) selected from O, S or N, wherein the ring can be optionally substituted with one or more substituents selected from alkyl, alkenyl, alkynyl, cycloalkyl, carboxy, alkoxy, aryloxy, halogen, aryl, amino, substituted amino, alkaryl, heteroaryl, heterocyclyl, heteroarylalkyl or heterocyclyl alkyl; and
R₄, R₅ and R₆ can be independently selected from hydrogen alkyl, aryl, heteroaryl, heterocyclyl, alkenyl, alkynyl, halogen, nitro, cyano, hydroxy, alkoxy, thioalkoxy, amino or substituted amino;
   with the proviso that when R₂ is hydrogen, R₃ cannot be hydrogen, alkaryl or heteroaryl alkyl; when R₂ is alkyl, R₃ cannot be alkaryl or heteroaryl alkyl; when R₂ is alkaryl, R₃ cannot be hydrogen or alkyl; when R₂ is heteroaryl alkyl, R₃ cannot be alkyl; when R₁ is alkyl, R₂ and R₃ cannot be hydrogen and alkyl, respectively; and when R₁ is hydrogen; R₂ and R₃ cannot be hydrogen and alkyl, respectively.

The following definitions apply to terms as used herein:

The term “alkyl,” unless otherwise specified, refers to a monoradical branched or unbranched saturated hydrocarbon chain having from 1 to 20 carbon atoms. This term can be exemplified by groups, for example, methyl, ethyl, n-propyl, iso-propyl, n-butyl, iso-butyl, t-butyl, n-hexyl, n-decyl, tetradecyl, and the like. Alkyl groups can be substituted with one or more substituents(s) selected from alkenyl, alkynyl, alkoxy, cycloalkyl, cycloalkenyl, acyl, acylamino, acyloxy, amino, substituted aminoaminocarbonyl, alkoxycarbonylamino, azido, cyano, halogen, hydroxy, oxo, thiocarbonyl, aryl, heterocyclyl, heteroaryl, arylthio, thiol, alkylthio, carboxy, carboxyalkyl, aryloxy, aminosulfonyl, aminocarbonylamino, hydroxyamino, alkoxyamino, —NHC(═O)R_{f}, —NR_{f}R_{q}, —C(═O)NR_{f}R_{q}, NHC(═O)NR_{f}R_{q}, —C(═O)heteroaryl, C(═O)heterocyclyl, —O—C(═O)NR_{f}R_{q} {wherein R_{f} and R_{q} can be independently selected from alkyl, alkenyl, cycloalkyl, cycloalkenyl, aryl, aralkyl, heterocyclyl, heteroaryl, heterocyclylalkyl, heteroarylalkyl}, nitro, and S(O)ₙR₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2). Unless otherwise constrained by the definition, all substituents can optionally be further substituted by 1-3 substituents chosen from alkyl, carboxy, carboxy-alkyl, aminocarbonyl, hydroxy, alkoxy, halogen, CF₃, amino, substituted amino, cyano, —OC(═O)NR_{f}R_{q}, —NHC(═O)NR_{f}R_{q} (wherein R_{f} and R_{q} can be independently selected from alkyl, alkenyl, cycloalkyl, cycloalkenyl, aryl, aralkyl, heterocyclyl, heteroaryl, heterocyclylalkyl or heteroarylalkyl) and —S(O)ₙR₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2); or an alkyl group as defined above can be interrupted by 1-5 atom(s) or groups independently chosen from oxygen, sulfur, keto, thiocarbonyl and —NR₈—(wherein R₈ can be hydrogen, alkyl, cycloalkyl, alkenyl, cycloalkenyl, alkynyl or aryl). Unless otherwise constrained by the definition, all substituents can optionally be further substituted by 1-3 substituents chosen from alkyl, carboxy, carboxyalkyl, aminocarbonyl, hydroxy, alkoxy, halogen, CF₃, amino, substituted amino, cyano, and —S(O)R₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2); or an alkyl group as defined above that can have substituents as defined above and can also be interrupted by 1-5 atoms or groups as defined above.

The term “alkenyl,” unless otherwise specified, refers to a monoradical branched or unbranched unsaturated hydrocarbon, having, for example, from 2 to 20 carbon atoms with cis or trans geometry. Particular alkenyl groups include ethenyl or vinyl (CH═CH₂), 1-propylene or allyl (—CH₂CH═CH₂), iso-propylene (—C(CH₃)═CH₂), bicyclo[2.2.1]heptene, and the like. In the event that an alkenyl group is attached to a heteroatom, the double bond cannot be alpha to the heteroatom. Alkenyl groups can be substituted with one or more substituents selected from alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, carboxy, carboxyalkyl, aryloxy, heterocyclyl, heteroaryl, aminosulfonyl, aminocarbonylamino, hydroxyamino, alkoxyamino, nitro and —S(O)ₙR₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2); or one or more carbon atom(s) can be replaced by keto or thiocarbonyl. Unless otherwise constrained by the definition, all substituents can optionally be substituted by 1-3 substituent(s) chosen from alkyl, carboxy, carboxyalkyl, aminocarbonyl, hydroxy, alkoxy, halogen, CF₃, amino, substituted amino, cyano, and —S(O)ₙR₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2).

The term “alkynyl,” unless otherwise specified, refers to a monoradical of an unsaturated hydrocarbon, having from 2 to 20 carbon atoms. Particular alkyl groups include ethynyl, (—C═CH), propargyl (or propynyl, —CH₂C═CH), and the like. In the event that an alkynyl group is attached to a heteroatom, the triple bond cannot be alpha to the heteroatom. An alkynyl group can be substituted with one or more substituents selected from alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, carboxy, carboxyalkyl, aryloxy, aminosulfonyl, aminocarbonylamino, hydroxyamino, alkoxyamino, nitro, heterocyclyl, heteroaryl, and —S(O)ₙR₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2); or one or more carbon atom can be replaced by keto or thiocarbonyl. Unless otherwise constrained by the definition, all substituents can optionally be substituted by 1-3 substituents chosen from alkyl, carboxy, carboxyalkyl, aminocarbonyl, hydroxy, alkoxy, halogen, CF₃, amino, substituted amino, cyano, and —S(O)ₙR₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2).

The term “cycloalkyl,” unless otherwise specified, refers to (un)saturated cyclic hydrocarbon of from 3 to 20 carbon atoms having a single cyclic ring or multiple condensed rings, which can optionally contain one or more olefinic bonds, unless otherwise constrained by the definition. Such cycloalkyl groups include, by way of example, single ring structures such as cyclopropyl, cyclobutyl, cyclopentyl, cyclooctyl, cyclopropylene, cyclobutylene and the like, or multiple ring structures such as adamantanyl and bicyclo[2.2.1]heptane, or cyclic alkyl groups to which is fused an aryl group, for example, indane, and the like. Spiro and fused ring structures can also be included. Cycloalkyl groups can be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{f}R_{q}, —NHC(═O)NR_{f}R_{q}, —NHC(═O)R_{f}, —C(═O)NR_{f}R_{q}, —O—C(═O)NR_{f}R_{q} (wherein R_{f} and R_{q} are independently selected from alkyl, alkenyl, cycloalkyl, cycloalkenyl, aryl, aralkyl, heterocyclyl, heteroaryl, heterocyclylalkyl or heteroarylalkyl), nitro, heterocyclyl, heteroaryl, heterocyclylalkyl, heteroarylalkyl, or S(O)ₙR₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2). Unless otherwise constrained by the definition, cycloalkyl substituents optionally can be substituted further by 1-3 substituents selected from alkyl, carboxy, hydroxy, alkoxy, halogen, CF₃, —NR_{f}R_{q}, —C(═O)NR_{f}R_{q}, —NHC(═O)NR_{f}R_{q}, —C(═O)NR_{f}R_{q} (wherein R_{f} and R_{q} can be independently selected from alkyl, alkenyl, cycloalkyl, cycloalkenyl, aryl, aralkyl, heterocyclyl, heteroaryl, heterocyclylalkyl or heteroarylalkyl), cyano or S(O)ₙR₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2).

“Substituted amino,” unless otherwise specified, refers to a group —N(R₈)₂ (wherein each R₈ can be independently selected from hydrogen (provided that both R₈ are not hydrogen), alkyl, alkenyl, alkynyl, alkaryl, cycloalkyl, aryl, heteroaryl, heterocyclyl, heterocyclylalkyl, heteroarylalkyl, S(O)ₙR₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2), C(═O)R₇ (wherein R₇ can be hydrogen, alkyl, alkenyl, alkynyl, cycloalkyl, aryl, heterocyclyl, heteroaryl, heteroarylalkyl or heterocyclylalkyl), or C(═O)OR₉ (wherein R₉ can be selected from alkyl, alkaryl, heteroarylalkyl, aryl, heteroaryl or heterocyclyl)). Unless otherwise constrained by the definition, all substituents can optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2).

The term “alkoxy” denotes the group O-alkyl wherein alkyl is the same as defined above.

The term “alkaryl” or “aralkyl,” unless otherwise specified, refers to (CH₂)ₚaryl, wherein p can be an integer in the range of 1-6 and aryl is as defined below. Examples of alkaryl include benzyl, ethylphenyl and the like.

The term “aryl,” unless otherwise specified, refers to carbocyclic aromatic groups, for example, phenyl, biphenyl or naphthyl systems and the like, optionally substituted with 1 to 3 substituents selected from halogen, hydroxy, alkyl, alkenyl, alkynyl, cycloalkyl, alkoxy, acyl, cycloalkoxy, CF₃, aryloxy, cyano, nitro, COORₑ (wherein Rₑ can be hydrogen, alkyl, alkenyl, cycloalkyl, aralkyl, heterocyclylalkyl or heteroarylalkyl), NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} can be independently selected from alkyl, alkenyl, cycloalkyl, cycloalkenyl, aryl, aralkyl, heterocyclyl, heteroaryl, heterocyclylalkyl or heteroarylalkyl), —S(O)ₙR₇ (wherein R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2), carboxy, heterocyclyl, heteroaryl, heterocyclylalkyl, heteroarylalkyl, amino carbonyl amino, —C(═O)R₁₀ (wherein R₁₀ can be hydrogen, alkyl, cycloalkyl, aryl, alkaryl, amino, substituted amino, hydroxy, alkoxy, heteroaryl, heterocyclyl or (CH₂)₀₋₃C(═O)N(R₈)₂ (wherein R₈ can be hydrogen, alkyl, cycloalkyl, alkenyl, cycloalkenyl, alkynyl or aryl)). The aryl group optionally can be fused with a cycloalkyl group can optionally contain one or more heteroatom selected from O, N or S.

The term “carboxy,” unless otherwise specified, refers to —C(═O)O—R₁₁ (wherein R₁ can be selected from hydrogen, alkyl, alkenyl, alkynyl or cycloalkyl).

The term “heteroaryl,” unless otherwise specified, refers to an aromatic ring structure containing 2 to 6 carbon atoms, or a bicyclic aromatic group having 4 to 10 carbon atoms, with one or more heteroatom(s) independently selected from N, O or S, optionally substituted with 1 to 3 substituent(s) selected from halogen, hydroxy, alkyl, alkenyl, alkynyl, cycloalkyl, carbamoyl, aryl, alkoxy, alkaryl, cyano, oxo, nitro, heterocyclyl, heteroaryl, optionally substituted amino (wherein the substituents are selected from alkyl, alkenyl, alkynyl, cycloalkyl, or aryl); carboxy, —C(═O)R₁ (wherein R₁₁ can be selected from hydrogen, alkyl, alkenyl, alkynyl or cycloalkyl), —C(═O)N(R₈)₂ (wherein R₈ can be hydrogen, alkyl, cycloalkyl, alkenyl, cycloalkenyl, alkynyl or aryl), —NR_{f}R_{q}, CH═NOH, CH₂)_{w}C(═O)R₈ {wherein w can be an integer from 0-4 and R₈ can be hydrogen, hydroxy, OR_{f}, NR_{f}R_{q}, —NHOR_{z} or —NHOH}, —C(═O)NR_{f}R_{q} or —NHC(═O)NR_{f}R_{q}, —S(O)ₙR₇, —O—C(═O)NR_{f}R_{q}, —O—C(═O)R_{f}, —O—C(═O)OR_{f} (wherein R₇ can be hydrogen, alkyl, alkenyl, alkynyl, cycloalkyl, aryl, heterocyclyl, heteroaryl, heteroarylalkyl or heterocyclylalkyl; n can be 0, 1 or 2; R_{f} and R_{q} can be independently selected from alkyl, alkenyl, cycloalkyl, cycloalkenyl, aryl, aralkyl, heterocyclyl, heteroaryl, heterocyclylalkyl or heteroarylalkyl; and R_{z} can be alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrrolyl, oxazolyl, thiazolyl, thienyl, isoxazolyl, triazinyl, furanyl, benzofuranyl, indolyl, benzothiazolyl, benzoxazolyl, and the like.

The term ‘heterocyclyl,” unless otherwise specified, refers to a non aromatic cycloalkyl group having 5 to 10 atoms in which 1 to 3 carbon atoms in a ring are replaced by heteroatoms selected from O, S and N, and are optionally benzofused or fused heteroaryl of 5-6 ring members and/or are optionally substituted wherein the substituents are selected from halogen, hydroxy, alkyl, alkenyl, alkynyl, cycloalkyl, acyl, carbamoyl, aryl, alkoxy, alkaryl, cyano, nitro, oxo, optionally substituted amino (wherein the substituents are selected from alkyl, alkenyl, alkynyl, cycloalkyl, aryl); carboxy, C(═O)R₁₀ (wherein R₁₀ can be hydrogen, alkyl, cycloalkyl, aryl, alkaryl, amino, substituted amino, hydroxy, alkoxy, heteroaryl, heterocyclyl or (CH₂)₀₋₃C(═O)N(R₈)₂ (wherein R₈ can be hydrogen, alkyl, cycloalkyl, alkenyl, cycloalkenyl, alkynyl or aryl)); C(═O)N(R₈)₂ (wherein R₈ can be hydrogen, alkyl, cycloalkyl, alkenyl, cycloalkenyl, alkynyl or aryl); heterocyclyl, heteroaryl, —O—C(═O)R_{f}, —O—C(═O)OR_{f}, —C(═O)NR_{f}R_{q}, S(O)ₙR₇, —O—C(═O)NR_{f}R_{q}, —NHC(═O)NR_{f}R_{q}, —NR_{f}R_{q} (wherein R_{f} and R_{q} can be independently selected from alkyl, alkenyl, cycloalkyl, cycloalkenyl, aryl, aralkyl, heterocyclyl, heteroaryl, heterocyclylalkyl, heteroarylalkyl; R₇ can be hydrogen, alkyl, alkenyl, alkynyl, cycloalkyl, aryl, heterocyclyl, heteroaryl, heteroarylalkyl or heterocyclylalkyl; and n can be 0, 1 or 2) or guanidine. Examples of heterocyclyl groups are oxazolidinyl, dihydroisoxazolyl, azabicyclohexyl, pyridinyl, isoindole-1,3-dione, piperidinyl, piperazinyl, benzoxazinyl, benzthiazinyl, benzimidazolyl, carbazolyl, indolyl, phenoxazinyl, phenothiazinyl, tetrahydrofuranyl, dihydrofuranyl, dihydropyridinyl, piperidinyl, piperazinyl, dihydrobenzofuryl, dihydroindolyl, and the like.

The term “heteroarylalkyl” refers to alkyl-heteroaryl group wherein the alkyl and heteroaryl are the same as defined earlier.

The term “heterocyclylalkyl” refers to alkyl-heterocyclyl group wherein the alkyl and heterocyclyl are the same as defined earlier.

The term “acyl” as defined herein refers to —C(═O)R₁₀ (wherein R₁₀ can be hydrogen, alkyl, cycloalkyl, aryl, alkaryl, amino, substituted amino, hydroxy, alkoxy, heteroaryl, heterocyclyl or (CH₂)₀₋₃C(═O)N(R₈)₂ (wherein R₈ can be hydrogen, alkyl, cycloalkyl, alkenyl, cycloalkenyl, alkynyl or aryl)). Examples of acyl include, for example, acetyl and benzoyl.

The term “halogen,” as defined herein, refers to F, Cl, Br or I.

The term “acyl,” as defined herein, refers to CORᵣ (wherein Rᵣ can be hydrogen, alkyl, cycloalkyl, aryl, aralkyl, heteroaryl, heterocyclyl, heteroarylalkyl, heterocyclylalkyl or substituted amino).

In accordance with yet another aspect, there are provided processes for the preparation of the compounds as described herein.

The compounds disclosed herein can be prepared by techniques well known in the art. In addition, these compounds can be prepared following illustrative reaction sequences as depicted in Schemes I, II and III.

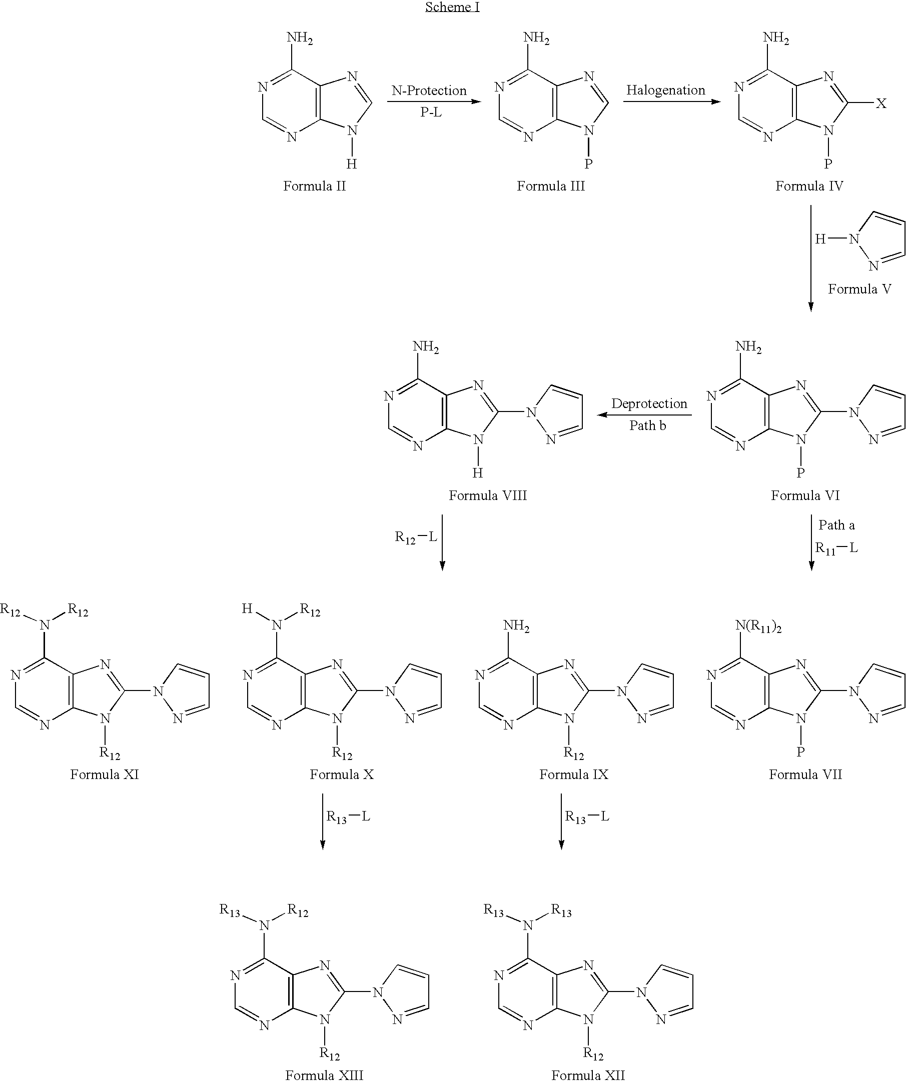

Compounds of Formulae VII can be prepared, for example, according to Scheme I (Path a). Thus, a compound of Formula II can be N-protected with a compound of Formula P-L (wherein P can be protecting group, such as alkaryl, and L can be leaving atom or group, such as Cl, Br, F, I) to form a compound of Formula III. A compound of Formula III can be halogenated to form a compound of Formula IV (wherein X can be halogen). A compound of Formula IV can be reacted with a pyrazole of Formula V to form a compound of Formula VI. A compound of Formula VI can be reacted with a compound of Formula R₁₁-L to form a compound of Formula VII (wherein R₁₁ represents R₃, and R₃ is the same as defined earlier).

The N-protection of a compound of Formula II to form a compound of Formula III can be carried out, for example, by following procedures described in J. Heterocyclic Chem. Vol. 19, 249-251 (1982), J. Heterocyclic Chem. Vol 1, 115-120 (1964), or Bioorg. Med. Chem. Vol 6, 523-533 (1998).

The halogenation of a compound of Formula III can be carried out in the presence of a halogenating agent, for example, N-bromosuccinimide, N-chlorosuccinimide, N-iodosuccinimide or a mixture thereof. The halogenation of a compound of Formula III can also be carried out in a solvent, for example, dimethylformamide, dimethylsulphoxide, tetrahydrofuran or a mixture thereof.

The reaction of a compound of Formula IV with a compound of Formula V to form a compound of Formula VI can be carried out in a solvent, for example, dimethylformamide, dimethylsulphoxide, tetrahydrofuran or a mixture thereof. The reaction of a compound of Formula IV with a compound of Formula V can also be carried out in the presence of a base, for example, sodium hydride, lithium hydride, lithium diisopropyl amide, sodium cyanoborohydride or a mixture thereof.

The reaction of a compound of Formula VI (path a) with a compound of Formula R₁₁-L to form a compound of Formula VII can be carried out in a solvent, for example, toluene, tetrahydrofuran, dimethylformamide, dimethylsulphoxide or a mixture thereof. The reaction of a compound of Formula VI with a compound of Formula R₁₁-L can also be carried out in the presence of a base, for example, pyridine, triethylamine, potassium carbonate, lithium hydride, sodium hydride or a mixture thereof.

The compound(s) prepared following Scheme I path a include, for example:
N-(9-Benzyl-8-pyrazol-1-yl-9H-purin-6-yl)-2,2-dimethylpropionamide (Compound No. 1)
N-Acetyl-N-(9-benzyl-8-pyrazol-1-yl-9H-purin-6-yl)acetamide (Compound No. 2)
N-benzoyl-N-(9-benzyl-8-pyrazol-1-yl-9H-purin-6-yl)benzamide (Compound No. 3)

Compounds of Formulae XI, XII or XIII can also be prepared, for example, according to Scheme I (Path b). Thus, deprotecting a compound of Formula VI forms a compound of Formula VIII. The compound of Formula VIII can be reacted with a compound of Formula R₁₂-L (wherein R₁₂ can be alkaryl and L can be leaving atom or group) to form at least one compound of:
Formula IX, which can be finally reacted with a compound of Formula R₁₃-L (wherein R₁₃ can be R₂ or R₃, but not hydrogen, and R₂ and R₃ are the same as defined earlier) to form a compound of Formula XII,
Formula X, which can be finally reacted with a compound of Formula R₁₃-L to form a compound of Formula XIII (wherein R₁₃ is the same as defined above), or
Formula XI (wherein R₁₂ is the same as defined above).

The deprotection of a compound of Formula VI (Path b) to form a compound of Formula VIII can be carried out following the procedure described in Protective Groups in Organic Synthesis, Greene et al., Third Edition, 1999, Wiley Interscience Publications, pp-579-580.

The reaction of a compound of Formula VIII with a compound of Formula R₁₃-L to form at least one compound of Formula IX, X or XI can be carried out, for example, by following procedures described in J. Heterocyclic Chem. Vol. 19, 249-251 (1982), J. Heterocyclic Chem. Vol. 1, 115-120 (1964) or Bioorg. Med. Chem. Vol. 6, 523-533 (1998).

The reaction of compound of Formula IX with a compound of Formula R₁₃-L to form a compound of Formula XII can be carried out in a solvent, for example, toluene, tetrahydrofuran, dimethylformamide, dimethylsulphoxide or a mixture thereof. The reaction of a compound of Formula IX with a compound of Formula R₁₃-L can also be carried out in the presence of a base, for example, pyridine, triethylamine, potassium carbonate, lithium hydride, sodium hydride or a mixture thereof.

The reaction of a compound of Formula X with a compound of Formula R₁₃-L to form a compound of Formula XIII can be carried out in a solvent, for example, toluene, tetrahydrofuran, dimethylformamide, dimethylsulphoxide or a mixture thereof. The reaction of a compound of Formula X with a compound of Formula R₁₃-L can also be carried out in the presence of a base, for example, pyridine, triethylamine, potassium carbonate, lithium hydride, sodium hydride or a mixture thereof.

The compounds prepared following Scheme I, path b include, for example:
Bis-(2-chlorobenzyl)-[9-(2-chlorobenzyl)-8-pyrazole-1-yl-9H-purin-6-yl]-amine (Compound No. 4)
1-(9-Benzyl-8-pyrazol-1-yl-9H-purin-6-yl)-3-(4-chlorophenyl)urea (Compound No. 6)
1-Allyl-3-(9-benzyl-8-pyrazol-1-yl-9H-purin-6-yl)-urea (Compound No. 12).

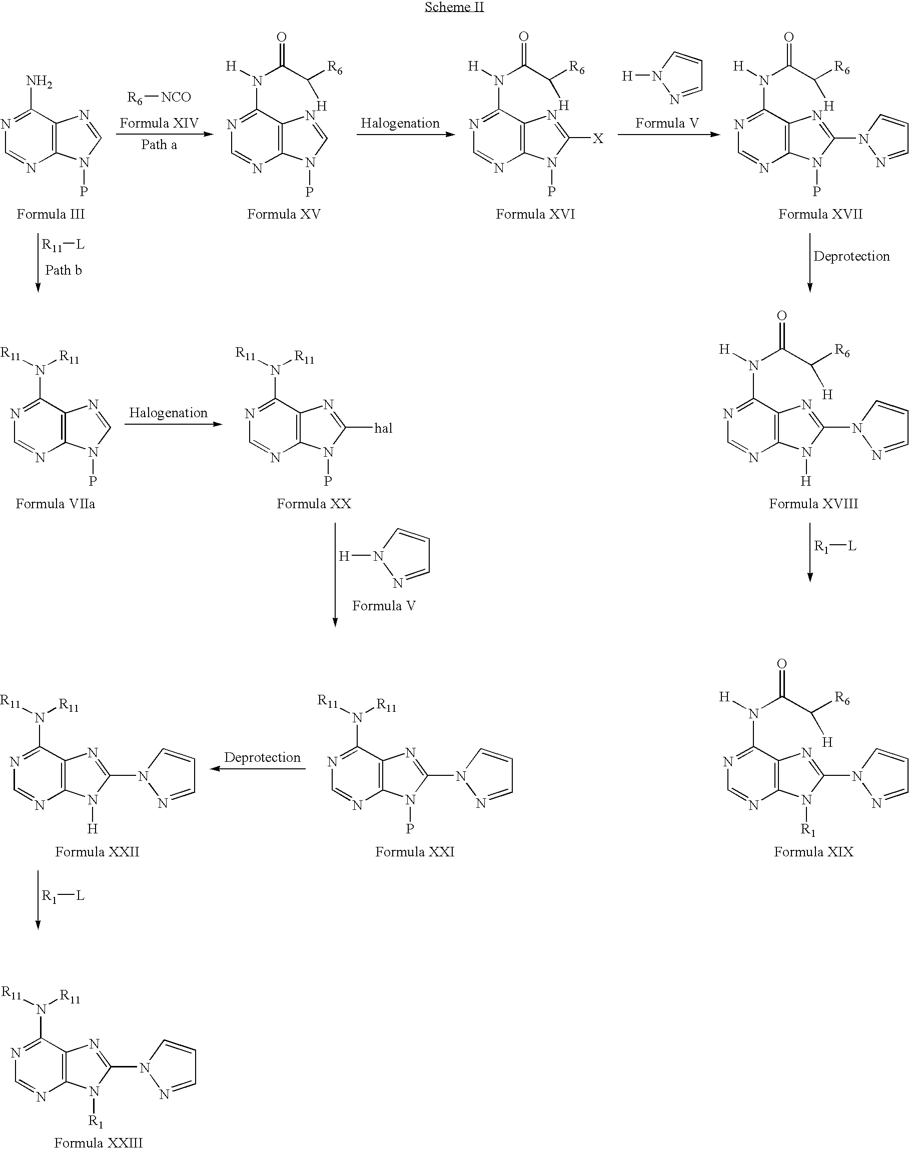

Compounds of Formula XIX can be prepared, for example, according to Scheme II (Path a). Thus, reacting a compound of Formula III with a compound of Formula XIV forms a compound of Formula XV (wherein P can be a protecting group and R₆ is the as defined earlier). The compound of Formula XV can be halogenated to form a compound of Formula XVI (wherein X can be halogen). The compound of Formula XVI can be reacted with pyrazole of Formula V to form a compound of Formula XVII. The compound of Formula XVII can be deprotected to form a compound of Formula XVIII. The compound of Formula XVIII can be reacted with a compound of Formula R₁-L to form a compound of Formula XIX (wherein R₁ is the same as defined earlier).

The reaction of a compound of Formula III with a compound of Formula XIV can be carried out in a solvent, for example, dichloromethane, dichloroethane, dimethylformamide or a mixture thereof. The halogenation of a compound of Formula XV to form a compound of Formula XVI can be carried out in a solvent, for example, dimethylformamide, dimethylsulphoxide, tetrahydrofuran or a mixture thereof. The halogenation of a compound of Formula XV to form a compound of Formula XVI can be carried out in the presence of a halogenating agent, for example, N-bromosuccinimide, N-chlorosuccinimide, N-iodosuccinimide or a mixture thereof.

The reaction of a compound of Formula XVI with a compound of Formula V can be carried out in a solvent, for example, dimethylformamide, dimethylsulphoxide, tetrahydrofuran or a mixture thereof. The reaction of a compound of Formula XVI with a compound of Formula V can be carried out in the presence of a suitable base, for example, sodium hydride, lithium hydride, sodium cyanoborohydride or a mixture thereof.

The deprotection of a compound of Formula XVII to form a compound of Formula XVIII can be carried out by following the procedure described in Protective Groups in Organic Synthesis, Greene et al., Third Edition, 1999, Wiley Interscience Publications, pp. 579-580.

The reaction of a compound of Formula XVIII with a compound of Formula R₁-L to form a compound of Formula XIX can be carried out, for example, by following procedures described in J. Heterocyclic Chem. Vol. 19, 249-251 (1982), J. Heterocyclic Chem. Vol. 1, 115-120 (1964) or Bioorg. Med. Chem. Vol. 6, 523-533 (1998).

Compounds of Formula XXIII can be prepared, for example, according to Scheme II (Path b). Thus, a compound of Formula III can be reacted with a compound of Formula R₁₁-L (wherein R₁₁ can be R₃ (wherein R₃ and L are the same as defined earlier) to form a compound of Formula VII (wherein P can be a protecting group as defined earlier and R₁₁ is as defined earlier). A compound of Formula R₁₁-L can be halogenated to form a compound of Formula XX (wherein hal can be halogen). A compound of Formula XX can be reacted with pyrazole of Formula V to form a compound of Formula XXI. A compound of Formula XXI can be deprotected to form a compound of Formula XXII. A compound of Formula XXII can be reacted with a compound of Formula R₁-L to form a compound of Formula XXIII (wherein R₁ is the same as defined earlier).

The reaction of a compound of Formula III with a compound of Formula R₁₁-L to form a compound of Formula VII can be carried out in the presence of a base, for example, pyridine, triethylamine, potassium carbonate, lithium hydride, sodium hydride or a mixture thereof.

The halogenation of a compound of Formula VII to form a compound of Formula XX can be carried out in a solvent, for example, dimethylformamide, dimethylsulphoxide, tetrahydrofuran or a mixture thereof. The halogenation of a compound of Formula VII to form a compound of Formula XX can be carried out in the presence of a halogenating agent, for example, N-bromosuccinimide, N-chlorosuccinimide, N-iodosuccinimide or a mixture thereof.

The reaction of a compound of Formula XX with pyrazole of Formula V to form a compound of Formula XXI can be carried out in a solvent, for example, dimethylformamide, dimethylsulphoxide, tetrahydrofuran or a mixture thereof. The reaction of a compound of Formula XX with pyrazole of Formula V can be carried out in a base, for example, sodium hydride, lithium hydride, sodium cyanoborohydride or a mixture thereof.

The deprotection of a compound of Formula XXI to form a compound of Formula XXII can be carried out, for example, by following the procedure described in Protective Groups in Organic Synthesis, Greene et al., Third Edition, 1999, Wiley Interscience Publications, pp-579-580.

The reaction of a compound of Formula XXII with a compound of Formula R₁-L to form a compound of Formula XXIII can be carried out, for example, following the procedure described in J. Heterocyclic Chem. Vol. 19, 249-251 (1982), J. Heterocyclic Chem., Vol. 1, 115-120 (1964) or Bioorg. Med. Chem. Vol. 6, 523-533 (1998).

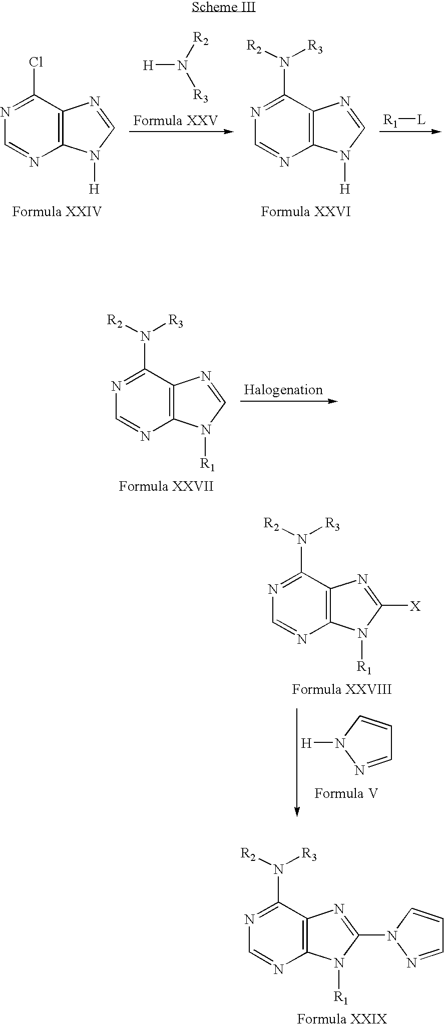

Compounds of Formula XXIX can be prepared, for example, according to Scheme III. Thus, reacting a compound of Formula XXIV with a compound of Formula XXV forms a compound of Formula XXVI (wherein R₂ and R₃ are the same as defined earlier). A compound of Formula XXVI can be reacted with a compound of Formula R₁-L to form a compound of Formula XXVII (wherein R₁ is the same as defined earlier). A compound of Formula XXVII can be halogenated to form a compound of Formula XXVIII (wherein X can be halogen). A compound of Formula XXVIII can be reacted with pyrazole of Formula V to form a compound of Formula XXIX (which is a compound of Formula I, wherein R₄, R₅ and R₆ are hydrogen).

The reaction of a compound of Formula XXVI with a compound of Formula R₁-L can be carried out, for example, following the procedure as described in J. Heterocyclic Chem. Vol. 19, 249-251 (1982), J. Heterocyclic Chem. Vol. 1, 115-120 (1964) or Bioorg. Med. Chem. Vol. 6, 523-533 (1998).

The halogenation of a compound of Formula XXVII to form a compound of Formula XXVIII can be carried out in a solvent, for example, dimethylformamide, dimethyl sulphoxide, tetrahydrofuran or a mixture thereof. The halogenation of compound of Formula XXVII to form a compound of Formula XXVIII can be carried out in the presence of a halogenating agent, for example, N-bromosuccinimide, N-chlorosuccinimide, N-iodosuccinimide or a mixture thereof.

The reaction of a compound of Formula XXVIII with pyrazole of Formula V to form a compound of Formula XXIX can be carried out in a solvent, for example, dimethylformamide, dimethylsulphoxidem, tetrahydrofuran or a mixture thereof. The reaction of a compound of Formula XXVIII with a pyrazole of Formula V can be carried out in the presence of a base, for example, sodium hydride, lithium hydride, sodium cyanoborohydride or a mixture thereof.

Compounds prepared following Scheme III include, for example:
(9-Benzyl-8-pyrazol-1-yl-9H-purin-6-yl)methylamine (Compound No. 5)
5-(6-Methylamino-8-pyrazol-1-yl-purin-9-yl-methyl)-oxazolidin-3-one (Compound No. 7)
9-[3-(3-Cyclopentyloxy-4-methoxy-phenyl)-5-methyl-4,5-dihydro-isoxazol-5-ylmethyl]-8-pyrazol-1-yl-9H-purin-6-yl}-methyl-amine (Compound No. 8)
(9-Benzyl-8-pyrazol-1-yl-9H-purin-6-yl)ethylamine (Compound No. 9)
[9-(2-Methoxybenzyl)-8-pyrazol-1-yl-9H-purin-6-yl]-methylamine (Compound No. 10)
[9-(2-Fluorobenzyl)-8-pyrazol-1-yl-9H-purin-6-yl]-methylamine (Compound No. 11)

Examples of particular compounds disclosed herein are given below in Table I.

TABLE I

| | |
|---|---|
| | Formula I |
| 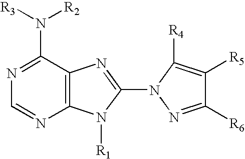 | |

(wherein R₄ = R₅ = R₆ = H)

| Compound No. | R₁ | R₂ | R₃ |
|---|---|---|---|
| 1. | 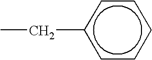 | H | 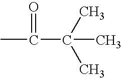 |
| 2. | 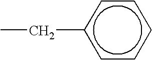 | 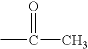 | 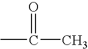 |
| 3. | 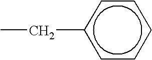 | 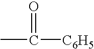 | 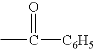 |
| 4. | 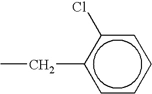 | 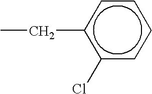 | 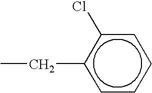 |
| 5. | 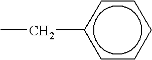 | H | CH₃ |
| 6. | 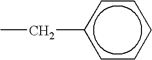 | H | 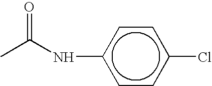 |
| 7. | 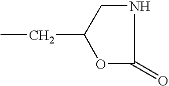 | H | CH₃ |
| 8. | 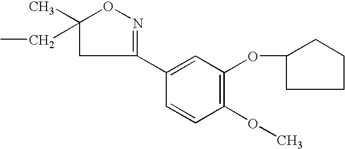 | H | CH₃ |
| 9. | 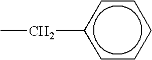 | H | C₂H₅ |
| 10 | 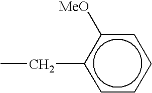 | H | CH₃ |
| 11 | 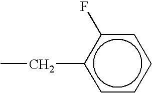 | H | CH₃ |
| 12 | 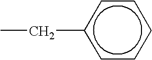 | H | 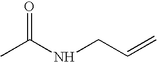 |

In the above schemes, where specific bases, condensing agents, reducing agents hydrolyzing agents, solvents, etc. are used, it is to be understood that other specific bases, condensing agents, reducing agents, hydrolyzing agents, solvents known to those skilled in the art can also be used. Similarly, the reaction temperature and duration of the reaction can be adjusted as desired.

### EXAMPLES

### Example 1

### Synthesis of methyl (9H-purin-6-yl)-amine

A solution of 6-chloropurine (0.1 g, 0.6472 mmol) in methylamine (1.5 mL) was stirred at 100° C. in an oil bath for 20 hours. The solvent of the resulting reaction mixture was evaporated off and a yellow semi-solid residue was obtained, which upon trituration with ethyl alcohol gave the title organic compound. Yield: 90 mg

### Example 2

### Synthesis of 9-benzyl-9H-purin-6-ylamine

To a suspension of adenine (3 g, 22.22 mmol) in benzene (55.5 mL) was added sodium hydroxide solution (9.8 mL of 10%) followed by the addition of tetra n-butyl ammonium bromide (1.430 g, 4.44 mmol). To the resulting reaction mixture, benzyl chloride (4.21 g, 3.8 mL, 33.3 mmol) was added under constant stirring. The reaction mixture was heated in an oil bath maintained at about 80-83° C. for 12 hours. The reaction mixture was cooled to room temperature to yield a crude organic compound, which was purified by column chromatography using methanol:ethyl acetate solvent mixture as an eluent. Yield=1.5 g.

### Example 3

### Synthesis of 9-benzyl-8-bromo-9H-purin-6-yl amine

To the solution of 9-benzyl-9H-purin-6-ylamine (0.15 g, 0.66 mmol, Example 2) in dry dimethylformamide (0.7 mL) was added N-bromosuccinimide (0.2373 g, 1.33 mmol). The reaction mixture was stirred for 2 hours at room temperature. Dimethylformamide was evaporated off under reduced pressure. The residue thus obtained was triturated by adding methanol (5-6 mL) to yield the title organic compound. Yield=0.14 g

### Example 4

### Synthesis of 9-benzyl-8-pyrazol-1-yl-9H-purin-6-ylamine

To the solution of pyrazole (0.3446 g, 5.065 mmol) and sodium hydride (0.13 g, 5.526 mmol) in dry dimethylformamide (0.7 mL) was added 9-benzyl-8-bromo-9H-purin-6-yl amine (0.14 g, 0.4605 mmol, Example 2). The reaction mixture was stirred at 100° C. for 22 hours. Dimethylformamide was evaporated off under reduced pressure. To the residue thus obtained, water (10 mL) was added. The organic compound was extracted with toluene (2×10 mL) and dried over sodium sulphate and subsequently concentrated under reduced pressure to yield the crude organic compound. The crude organic compound thus obtained was triturated with methanol to yield the title organic compound. Yield=0.1 g

### Example 5

### Synthesis of 8-pyrazol-1-yl-9H-purin-6-ylamine

The 9-benzyl-8-pyrazol-1-yl-9H-purin-6-ylamine (1.5 g, 5.1546 mmol, Example 4) was taken in dry methanol in formic acid solution (90%, 3.04 mL). To it ammonium formate (6.0938 g) was added, followed by addition of palladium on carbon (4.57 g, 10%) under nitrogen atmosphere. The reaction mixture was stirred for 30 minutes at room temperature, followed by refluxing at 50-55° C. for 24 hours. The palladium on carbon was filtered and the black solid thus obtained was washed with 200 mL of hot methanol. Methanol was evaporated off under reduced pressure to afford an organic compound, which was finally treated with brine, filtered, concentrated and dried to yield the title organic compound. Yield=0.64 g

### Example 6

### Synthesis of Bis-(2-chlorobenzyl)-[9-(2-chlorobenzyl)-8-pyrazol-1-yl-9H-purin-6-yl]-amine (Compound No. 4)

To a solution of 8-pyrazol-1-yl-9H-purin-6-ylamine (0.05 g, 0.248 mmol, Example 5) in dry dimethylformamide (0.5 mL) was added potassium carbonate (0.1373 g, 0.995 mmol). To the resulting reaction mixture was added 2-chlorobenzylbromide (0.0102 g, 0.4975 mmol) and the reaction was allowed to stir for about 16 hours at 110° C. The reaction mixture was diluted with methanol. The inorganic salts thus separated were filtered and washed with methanol. The filtrate was concentrated to dryness to yield the crude organic compound. The crude organic compound was purified over column chromatography by using methanol:ethyl acetate solvent mixture as an eluent to yield title organic compound. Yield=27 mg.

¹HNMR (CDCl₃): δ 8.38 (s, 1H), 8.00 (s, 1H), 7.75 (s, 1H), 7.18-7.36 (m, 12H), 6.40 (s, 1H), 5.87 (s, 2H), 5.78 (s, 2H), 5.08 (s, 2H)

Mass (M⁺+1): m/z 574.5

### Example 7

### Synthesis of 1-(9-Benzyl-8-pyrazol-1-yl-9H-purin-6-yl)-3-(4-chlorophenyl)urea (Compound No. 6)

To a solution of 9-benzyl-8-pyrazol-1-yl-9H-purin-6-ylamine (0.07 g, 0.2405 mmol, Example 4) in dichloroethane (0.3 mL) and dimethylformamide (0.3 mL) was added 4-chlorophenyl isocyanate (0.0369 g, 0.240 mmol). The resulting reaction mixture was stirred at room temperature for 2 hours. The solid compound thus obtained was filtered off and washed with dichloroethane. The product was purified by recrystallization from methanol. Yield=40 mg.

m.p.: 232-233° C.

¹HNMR (CDCl₃): δ 8.65 (s, 1H), 8.32 (s, 1H), 7.85 (s, 1H), 7.22-7.34 (m, 9H), 6.54 (s, 1H), 6.05 (s, 2H), 7.59-7.26 (d, 2H)

Mass (M⁺+1): m/z 445.14

By following the same procedure and by its using the suitable intermediates the following compounds were obtained
1-Allyl-3-(9-benzyl-8-pyrazol-1-yl-9H-purin-6-yl)-urea (Compound No. 12)

m.p.: 189.5-199° C.

Mass (M⁺+1): m/z 375.16

### Example 8

### Synthesis of N-benzoyl-N-(9-benzoyl-8-pyrazol-1-yl-9H-purin-6-yl)-benzamide (Compound No. 3)

To a solution of 9-benzyl-8-pyrazol-1-yl-9H-purin-6-ylamine (0.1027 g, 0.35 mmol, Example 4) in pyridine (0.5 mL) was added benzoylchloride (1.2 mL, 1.05 mmol) and the solution was heated in an oil bath maintained at about 80-85° C. for 40 minutes. Toluene was added to the resulting reaction mixture followed by removal of pyridine under reduced pressure. To the residue thus obtained was added aqueous sodium bicarbonate solution and the organic compound was extracted with chloroform (2×15 mL). The organic layer was dried over sodium sulphate, concentrated and dried to give an oily residue, which was finally treated with ether to yield the title organic compound. Yield=80 mg

m.p.: 197-198° C.

¹HNMR (CDCl₃): δ 8.70 (s, 1H), 8.30 (s, 1H), 8.13 (s, 1H), 7.24-7.86 (m, 15H), 6.45 (s, 1H), 6.05 (s, 2H)

Mass (M⁺+1): m/z 500.4

By following the same procedure and by its using the suitable intermediates the following compounds were obtained.
N-(9-Benzyl-8-pyrazol-1-yl-9H-purin-6-yl)-2,2-dimethyl propionamide (Compound No. 1)

m.p.: 124-125° C.

¹HNMR (CDCl₃): δ 8.80 (s, 1H), 8.35 (s, 1H), 7.85 (s, 1H), 7.21-7.26 (m, 5H), 6.02 (s, 2H), 1.41 (s, 9H)

Mass (M⁺+1): m/z 376.3
N-Acetyl-N-(9-benzyl-8-pyrazol-1-yl-9H-purin-6-yl)-acetamide (Compound No. 2)

¹HNMR (CDCl₃): δ 8.96 (s, 1H), 8.43 (s, 1H), 7.87 (s, 1H), 7.18-7.37 (m, 5H), 6.53 (s, 1H), 6.02 (s, 2H), 2.293 (s, 6H)

Mass (M⁺+1): m/z 376.5

### Example 9

### Synthesis of (9-Benzyl-8-pyrazol-1-yl-9H-purin-6-yl)methyl amine (Compound No. 5)

### Step a: Synthesis of methyl-(9-benzyl-9H-purin-6-yl)amine

The title compound was prepared following the procedure as described in Example 2 by using methyl-(9H-purin-6-ylamine) (Example 1) in place of adenine.

### Step b: Synthesis of methyl-(9-benzyl-8-bromo-9H-purin-6-yl)amine

The title compound was prepared following the procedure as described Example 3 by using the compound obtained from step a above in place of compound prepared in Example 2.

### Step c: (9-Benzyl-8-pyrazol-1-yl-9H-purin-6-yl)methylamine

The title organic compound was prepared following the procedure as described in Example 4 by using the compound obtained from step b above in place of 9-benzyl-8-bromo-9H-purin-6-ylamine.

m.p.: 115° C.

¹HNMR (CDCl₃): δ 8.42-8.43 (d, 1H), 7.96-7.99 (d, 1H), 7.63 (s, 1H), 7.35-7.42 (m, 5H), 6.74 (s, 1H), 6.43-6.45 (t, 1H), 5.58 (s, 2H), 3.19 (s, 3H)

Mass (M⁺+1): m/z 306.20

By following the same procedure and by utilizing the suitable intermediates the following compound(s) are also obtained.
5-(6-Methylamino-8-pyrazol-1-yl-purin-9-ylmethyl)-oxazolidin-3-one (Compound No. 7)
9-[3-(3-Cyclopentyloxy-4-methoxy-phenyl)-5-methyl-4,5-dihydro-isoxazol-5-ylmethyl]-8-pyrazol-1-yl-9H-purin-6-yl-methyl amine (Compound No. 8)
[9-(2-Methoxybenzyl)-8-pyrazol-1-yl-9H-purin-6-yl]-methylamine (Compound No. 10)

¹H NMR (CDCl₃): 8.42 (s, 1H), 8.13 (s, 1H), 7.75 (s, 1H), 7.56-7.54 (d, 1H), 7.37-7.32 (t, 1H), 6.95-6.91 (d, 2H), 5.57 (s, 2H), 3.88 (s, 1H), 2.95 9s, 3H)

Oil, Mass (M⁺+1): m/z 336.31
[9-(2-Fluorobenzyl)-8-pyrazol-1-yl-9H-purin-6-yl]-methylamine (Compound No. 11)

8.43 (s, 1H), 8.12 (s, 1H), 7.76 (s, 1H), 7.63-7.58 (t, 1H), 7.34-7.32 (d, 1H), 7.16-7.12 (m, 2H), 6.45 (s, 1H), 5.62 (s, 2H), 3.21 (s, 3H)

Oil, Mass (M⁺+1): m/z 324.26

### Example 10

### Synthesis of (9-Benzyl-8-pyrazol-1-yl-9H-purin-6-yl)ethylamine (Compound No. 9)

### Step a: Synthesis of ethyl-(9-benzyl-9H-purin-6-yl)amine

The title compound was prepared following the procedure as described in Example 2 by using ethyl-(9H-purin-6-ylamine) (Example 1, by using ethyl amine in place of methyl amine)) in place of adenine.

### Step b: Synthesis of ethyl-(9-benzyl-8-bromo-9H-purin-6-yl)amine

The organic compound was prepared following the procedure as described Example 3 by using compound obtained from step a above in place of compound prepared in Example 2.

### Step c: Synthesis of (9-Benzyl-8-pyrazol-1-yl-9H-purin-6-yl)methylamine

The title organic compound was prepared following the procedure as described in example 4 by using compound obtained from step b above in place of 9-benzyl-8-bromo-9H-purin-6-ylamine.

¹HNMR (CDCl₃): 8.5 (s, 1H), 8.11-8.09 (d, 1H), 7.31-7.18 (ArH, 5H), 6.5 (s, 1H), 5.59-5.56 (q, 2H), 3.15-3.12 (d, 2H), 0.92-0.85 (t, 3H)

Mass (M⁺+1): m/z 320.34

### Example 11

### Efficacy of Compounds as PDE IV Inhibitors

### PDE-IV Enzyme Assay

The efficacy of compounds of PDE-4 inhibitors was determined by an enzyme assay using U937 cell cytosolic fraction (BBRC, 197: 1126-1131, 1993). Hydrolysis of cAMP to AMP was monitored using HPLC and [³H]cAMP in the sample was detected using FLO-ONE Detector.

The enzyme preparation was incubated in the presence and absence of the test compound for 30 min and amount of [3H]cAMP was measured in the sample. IC₅₀ valves for compounds tested are found to be in the range of from about 1 mmol to about 10 nmol.

Compounds described herein were tested using this assay and the compounds exhibited IC₅₀ values of between about 2 μM to greater than about 10 μM, and in some instances, from about 2.5 μM to about 7 μM, and even from 3 μM to about 5 μM.

We claim:

1. A compound having the structure of Formula I, 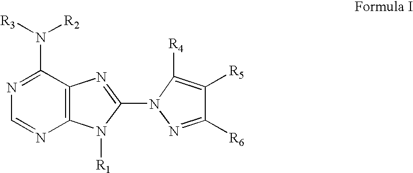
their pharmaceutically acceptable salts, pharmaceutically acceptable solvates, enantiomers, diastereomers or N-oxides wherein
R₁ is hydrogen, alkyl, cycloalkyl, aryl, alkaryl, heteroaryl, heteroaryl alkyl, or heterocyclyl alkyl;
R₂ and R₃ independently are hydrogen, alkyl, alkenyl, alkynyl, acyl, alkaryl, heteroaryl alkyl, or heterocyclyl alkyl;
R₂ and R₃ together join to form three to eight membered cyclic rings, which is optionally benzofused containing 0-3 heteroatom(s) selected from O, S or N, wherein the ring is optionally substituted with one or more substituents selected from alkyl, alkenyl, alkynyl, cycloalkyl, carboxy, alkoxy, aryloxy, halogen, aryl, amino, substituted amino, alkaryl, heteroaryl, heterocyclyl, heteroarylalkyl or heterocyclyl alkyl; and
R₄, R₅ and R₆ are independently selected from hydrogen alkyl, aryl, heteroaryl, heterocyclyl, alkenyl, alkynyl, halogen, nitro, cyano, hydroxy, alkoxy, thioalkoxy, amino, or substituted amino;
with the provisos that when R₂ is hydrogen, R₃ cannot be hydrogen, alkaryl or heteroaryl alkyl; when R₂ is alkyl, R₃ cannot be alkaryl or heteroaryl alkyl; when R₂ is alkaryl, R₃ cannot be hydrogen or alkyl; when R₂ is heteroaryl alkyl, R₃ cannot be alkyl; when R₁ is alkyl, R₂ and R₃ cannot be hydrogen and alkyl, respectively; and when R₁ is hydrogen; R₂ and R₃ cannot be hydrogen and alkyl, respectively.

2. The compound according to claim 1, wherein R₁ is aralkyl.

3. The compound according to claim 2, wherein R₁ is benzyl, 2-chlorobenzyl, 2-fluorobenzyl or 2-methoxybenzyl.

4. The compound according to claim 1, wherein R₂ is hydrogen, acyl or aralkyl.

5. The compound according to claim 4, wherein R₂ is acetyl, benzoyl or 2-chlorobenzyl.

6. The compound according to claim 1, wherein R₃ is alkyl, acyl or aralkyl.

7. The compound according to claim 6, wherein R₃ is methyl, ethyl, COCH₃, COC(CH₃)₃, COC₆H₅, CONH(4-chlorophenyl), CONHCH₂CH═CH₂ or 2-chlorobenzyl.

8. The compound according to claim 1, wherein R₄, R₅ and R₆ are hydrogen.

9. A compound which is
N-(9-Benzyl-8-pyrazol-1-yl-9H-purin-6-yl)-2,2-dimethylpropionamide,
N-Acetyl-N-(9-benzyl-8-pyrazol-1-yl-9H-purin-6-yl)acetamide,
N-benzoyl-N-(9-benzyl-8-pyrazol-1-yl-9H-purin-6-yl)benzamide,
Bis-(2-chlorobenzyl)-[9-(2-chlorobenzyl)-8-pyrazole-1-yl-9H-purin-6-yl]-amine,
(9-Benzyl-8-pyrazol-1-yl-9H-purin-6-yl)methylamine,
1-(9-Benzyl-8-pyrazol-1-yl-9H-purin-6-yl)-3-(4-chlorophenyl)urea,
1-Allyl-3-(9-benzyl-8-pyrazol-1-yl-9H-purin-6-yl)-urea,
[9-(2-Methoxybenzyl)-8-pyrazol-1-yl-9H-purin-6-yl]-methylamine,
[9-(2-Fluorobenzyl)-8-pyrazol-1-yl-9H-purin-6-yl]-methylamine,
(9-Benzyl-8-pyrazol-1-yl-9H-purin-6-yl)ethylamine or
their pharmaceutically acceptable salts, pharmaceutically acceptable solvates, enantiomers, diastereomers or N-oxides.

10. A pharmaceutical composition comprising a therapeutically effective amount of at least one compound of claim 1 together with at least one pharmaceutically acceptable carrier, excipient or diluent.

11. A method for treating, preventing, inhibiting or suppressing an inflammatory condition or disease in a patient, comprising administering to the said patient a therapeutically effective amount of at least one compound of claim 1.

12. A method for treating, preventing, inhibiting or suppressing an inflammatory condition or disease in a patient, comprising administering to the said patient a therapeutically effective amount of a pharmaceutical composition of claim 10.

13. A method for the treatment, prevention, inhibition or suppression of AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis or other inflammatory diseases in a patient comprising administering to said patient a therapeutically effective amount of at least one compound of the claim 1.

14. A method for the treatment, prevention, inhibition or suppression of AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis or other inflammatory diseases in a patient comprising administering to said patient a therapeutically effective amount of a pharmaceutical composition of claim 10.

15. A method for the preparation of compounds of Formula VII, 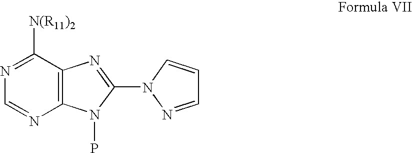
their pharmaceutically acceptable salts, pharmaceutically acceptable solvates, enantiomers, diastereomers or N-oxides, which method comprises the steps of:
a) N-protecting a compound of Formula II 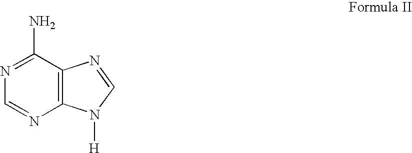
with a compound of Formula P-L to form a compound of Formula III, 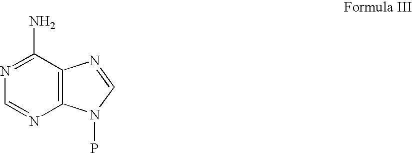
b) halogenating a compound of Formula III to form a compound of Formula IV, 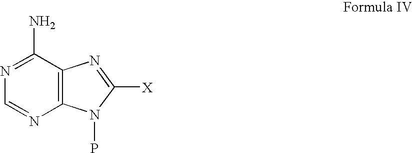
c) reacting a compound of Formula IV with pyrazole to form a compound of Formula VI, 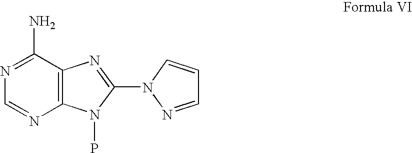
and
d) reacting a compound of Formula VI with a compound of Formula R₁-L to form a compound of Formula VII,
wherein P is a protecting group; L is a leaving atom or group; X is a halogen; and R₁₁ is R₃ (wherein R₃ is hydrogen, alkyl, alkenyl, alkynyl, acyl, alkaryl, heteroaryl alkyl, or heterocyclyl alkyl).

16. A method for the preparation of compounds of Formula XI, 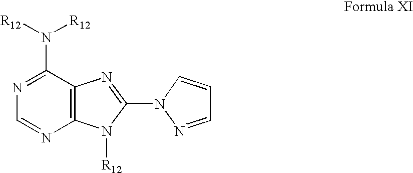
their pharmaceutically acceptable salts, pharmaceutically acceptable solvates, enantiomers, diastereomers or N-oxides, which method comprises the steps of:
a) deprotecting a compound of Formula VI 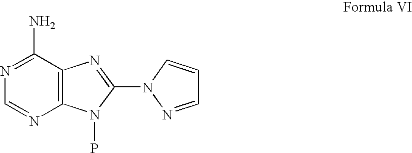
to form a compound of Formula VIII, 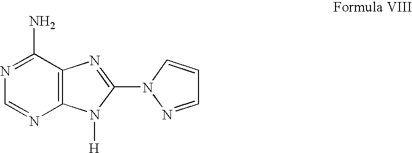
and
b) reacting a compound of Formula VIII with a compound of Formula R₁₂-L to form a compound of Formula XI
wherein P is a protecting group, L is a leaving atom or group and R₁₂ is aralkyl.

17. A method for the preparation of compounds of Formula XII, 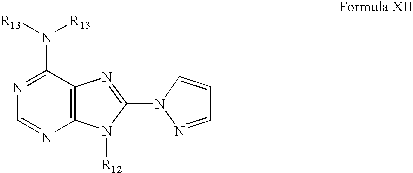
their pharmaceutically acceptable salts, pharmaceutically acceptable solvates, enantiomers, diastereomers or N-oxides, which method comprises the steps of:
a) reacting a compound of Formula VIII, 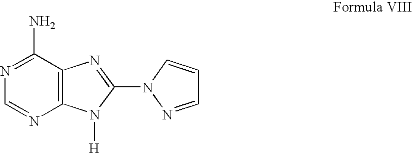
with a compound of Formula R₁₂-L to give a compound of Formula IX, 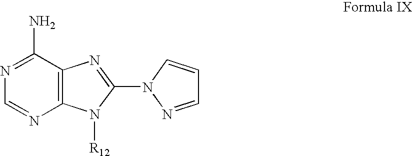
and
b) reacting a compound of Formula I with a compound of Formula R₁₃-L to form a compound of Formula XII,
wherein L is a leaving atom or group, R₁₂ is aralkyl and R₁₃ is R₂ or R₃ (wherein R₂ or R₃ independently is hydrogen, alkyl, alkenyl, alkynyl, acyl, alkaryl, heteroaryl alkyl, or heterocyclyl alkyl).

18. A method for the preparation of compounds of Formula XIII, 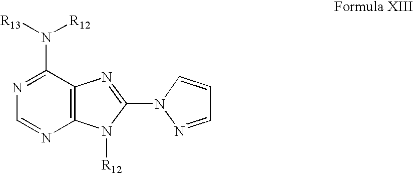
their pharmaceutically acceptable salts, pharmaceutically acceptable solvates, enantiomers, diastereomers or N-oxides, which method comprises the steps of:
a) reacting a compound of Formula VIII, 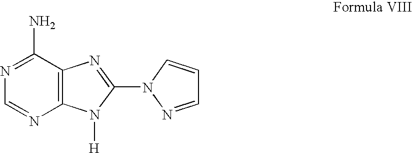
with a compound of Formula R₁₂-L to form a compound of Formula X, 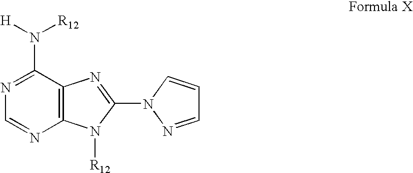
b) reacting a compound of Formula X with a compound of Formula R₁₃-L to form a compound of Formula XI,
wherein L is a leaving atom or group, R₁₂ is aralkyl, and R₁₃ is R₂ or R₃ (wherein R₂ or R₃ independently is hydrogen, alkyl, alkenyl, alkynyl, acyl, alkaryl, heteroaryl alkyl, or heterocyclyl alkyl).

19. A method for the preparation of compounds of Formula XIX, 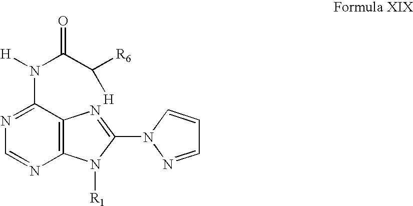
their pharmaceutically acceptable salts, pharmaceutically acceptable solvates, enantiomers, diastereomers or N-oxides, which method comprises the steps of:
a) reacting a compound of Formula III 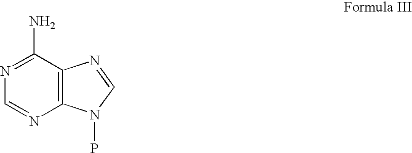
with a compound of Formula XIV,
R₆—NCO Formula XIV
to form a compound of Formula XV, 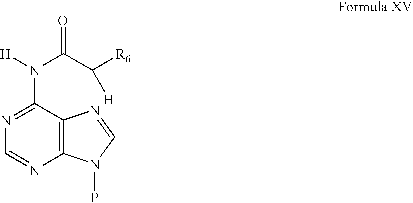
b) halogenating a compound of Formula XV to form a compound of Formula XVI, 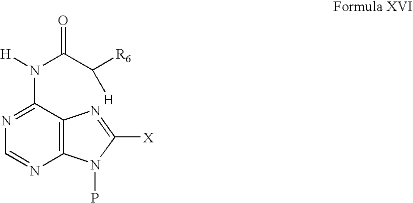
c) reacting a compound of Formula XVI with pyrazole gives a compound of Formula XVII, 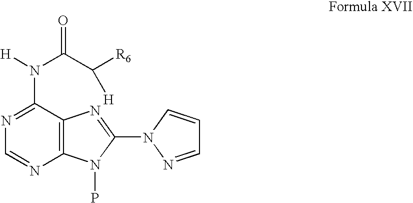
d) deprotecting a compound of Formula XVII to form a compound of Formula XVII, 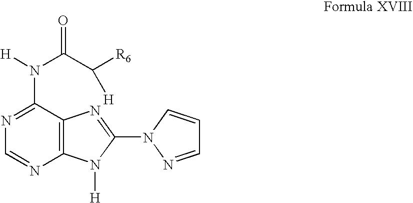
and
e) reacting a compound of Formula XVIII with a compound of Formula R₁-L to form a compound of Formula XIX,
wherein P is a protecting group; R₆ is hydrogen alkyl, aryl, heteroaryl, heterocyclyl, alkenyl, alkynyl, halogen, nitro, cyano, hydroxy, alkoxy, thioalkoxy, amino, or substituted amino; X is a halogen; L is leaving atom or group; and R₁ is hydrogen, alkyl, cycloalkyl, aryl, alkaryl, heteroaryl, heteroaryl alkyl, or heterocyclyl alkyl.

20. A method for the preparation of compounds of Formula XXIII, 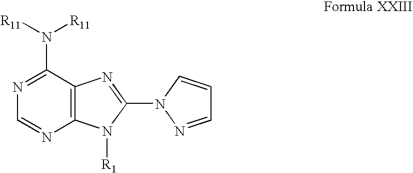
their pharmaceutically acceptable salts, pharmaceutically acceptable solvates, enantiomers, diastereomers or N-oxides, which method comprises the steps of:
a) reacting a compound of Formula III with a compound of Formula R₁₁-L 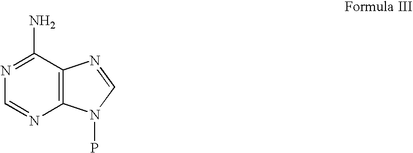
to form a compound of Formula VIIa, 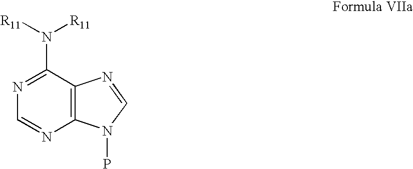
b) halogenating a compound of Formula VIIa to form a compound of Formula XX, 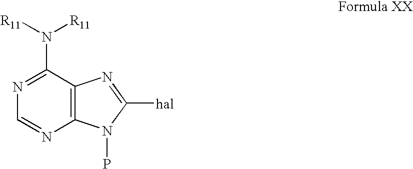
c) reacting a compound of Formula XX with pyrazole to form a compound of Formula XXI, 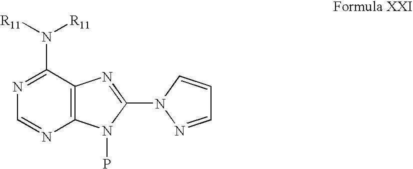
d) deprotecting a compound of Formula XXI to form a compound of Formula XXII, 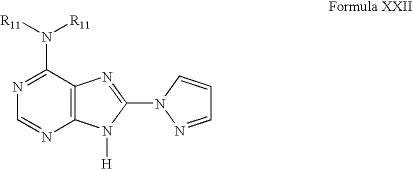
and
e) reacting a compound of Formula XXI with a compound of Formula R₁-L to form a compound of Formula XXII,
wherein P is a protecting group; L is leaving atom or group; R₁₁ is R₃ (wherein R₃ is hydrogen, alkyl, alkenyl, alkynyl, acyl, alkaryl, heteroaryl alkyl, or heterocyclyl alkyl); hal is halogen; and R₁ is hydrogen, alkyl, cycloalkyl, aryl, alkaryl, heteroaryl, heteroaryl alkyl, or heterocyclyl alkyl.

21. A method for the preparation of compounds of Formula XXIX, 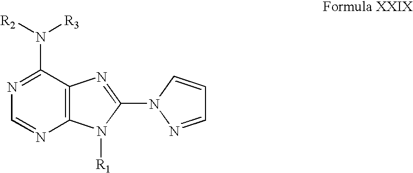
their pharmaceutically acceptable salts, pharmaceutically acceptable solvates, enantiomers, diastereomers or N-oxides, which method comprises the steps of:
a) reacting a compound of Formula XXIV 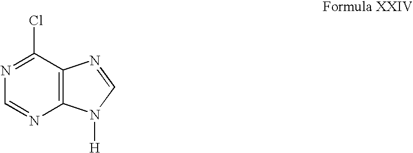
with a compound of Formula R₂R₃NH to form a compound of Formula XXVI, 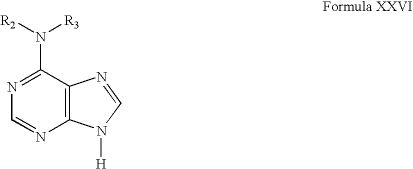
b) reacting a compound of Formula XXVI with a compound of Formula R₁-L to form a compound of Formula XXVII, 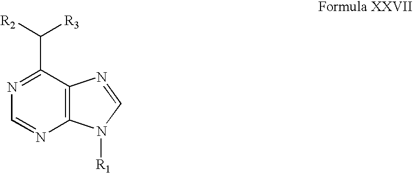
c) halogenating a compound of Formula XXVII to form a compound of Formula XXVIII, 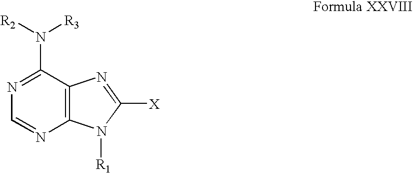
and
d) reacting a compound of Formula XXVIII with pyrazole to form a compound of Formula XXIX
wherein R₁ is hydrogen, alkyl, cycloalkyl, aryl, alkaryl, heteroaryl, heteroaryl alkyl, or heterocyclyl alkyl; and R₂ and R₃ independently is hydrogen, alkyl, alkenyl, alkynyl, acyl, alkaryl, heteroaryl alkyl, or heterocyclyl alkyl; L is a leaving atom or group; and X is a halogen.
